(19) United States
(12) Patent Application Publication
(10) Pub. No.: US 2017/0086754 A1
(43) Pub. Date: Mar. 30, 2017

(54) SYSTEM AND METHOD FOR MEASURING AND DETECTING PHYSIOLOGICAL APPEARANCES IN HUMANS

(71) Applicants: Waldo CONCEPCION, Menlo Park, CA (US); Yizhak Zloter, Holon (IL); Rosie Salcedo, Menlo Park, CA (US); Menachem Erad, Ashdod (IL)

(72) Inventors: Waldo CONCEPCION, Menlo Park, CA (US); Yizhak Zloter, Holon (IL); Rosie Salcedo, Menlo Park, CA (US); Menachem Erad, Ashdod (IL)

(21) Appl. No.: 15/275,491

(22) Filed: Sep. 26, 2016

Related U.S. Application Data
(60) Provisional application No. 62/233,317, filed on Sep. 26, 2015.

(51) Int. Cl. A61B 5/00; A61B 5/1171; A61B 5/1455; A61B 5/145; A61B 5/021; A61B 5/024

(52) U.S. Cl. CPC A61B 5/7275; A61B 5/0002; A61B 5/6814; A61B 5/6824; A61B 5/021; A61B 5/742; A61B 5/7455; A61B 5/024; A61B 5/14551; A61B 5/14532; A61B 5/0008; A61B 5/7405; A61B 5/1171; A61B 5/0022; A61B 5/681; A61B 5/6807; A61B 5/6804; A61B 5/6803; A61B 5/746

(57) ABSTRACT

The subject matter discloses a method, comprising collecting information from at least one blood pressure sensor attached to a person, wherein said collected information is related to the blood pressure conditions of the person, transmitting said collected information to an analysis system, said analysis system has access to prior blood pressure measurements of the person, transmitting the person's identity to said analysis system, the person is associated with said transmitted collected information, comparing said collected information with historical blood pressure information of said person, detecting an abnormal trend according to said comparison of the collected information with the historical information related to the blood pressure conditions of said person, generating an indication according to predefined procedures in case the abnormal trend is detected.

### FIELD OF THE INVENTION

The present invention relates to a blood-pressure measuring in general and more specifically to a method for measuring continuous blood pressure in persons.

### BACKGROUND OF THE INVENTION

Blood pressure varies depending on situation, activity, and disease states. It is regulated by the nervous and endocrine systems. Blood pressure that is low due to a disease state is called hypotension, and pressure that is consistently high is hypertension. Both have many causes ranging from mild to severe conditions. Both may be of sudden onset or of long duration. Long term high blood pressure is a risk factor for many diseases, including heart disease, stroke and kidney failure. Long term high blood pressure is more common than long term low blood pressure in Western countries. There are various devices for measuring the blood pressure of subjects in a continuous and in a one-time manner. In some cases, measuring blood pressure in real-time takes place without any interruptions (continuously) and without cannulating the human body (noninvasive). Measuring the continuous blood pressure can provide a good analysis at the point in time and the fashion of the changes in the blood pressure. However, known blood pressure measurements face difficulties in identifying the root cause to the changes in the blood pressure, and are unable to predict blood pressure incidents.

### SUMMARY OF THE INVENTION

It is an object of the present invention to disclose a method, comprising collecting information from at least one blood pressure sensor attached to a person, wherein said collected information is related to the blood pressure conditions of the person, transmitting said collected information to an analysis system, said analysis system has access to prior blood pressure measurements of the person, transmitting the person's identity to said analysis system, the person is associated with said transmitted collected information, comparing said collected information with historical blood pressure information of said person, detecting an abnormal trend according to said comparison of the collected information with the historical information related to the blood pressure conditions of said person, generating an indication according to predefined procedures in case the abnormal trend is detected, and storing said newly collected information in the analysis system.

In some cases, the method further comprises transmitting a date and a time of said information collection to the analysis system. In some cases, the method further comprises transmitting a physical position of said person during collection of the information to the analysis system. In some cases, the method further comprises collecting additional physiological data of the person. In some cases, the method further comprises transmitting information about the person's movements during collection of the information to the analysis system. In some cases, the method further comprises securing the identity of the person associated with the collected blood pressure information.

It is an object of the present invention to disclose a computerized device integrated with at least one blood pressure sensor, comprising an independent power source, a processor to collect blood pressure information from the at least one blood pressure sensor, a memory unit to store the information collected by the processor, a communication device, and a control system designed to associate the collected information with the person identity, transmit the collected information to a remote analysis system, receive feedback from said remote analysis system, and provide an indication to said person according to said received feedback.

In some cases, the computerized device further comprises sensors for sensing the physical position of said person while collecting the blood pressure information.

In some cases, the computerized device further comprises a display unit. In some cases, the display unit is used by said control system to display alerts to said person.

In some cases, the communication device is a wireless communication device. In some cases, the communication device is a wired communication device. In some cases, the computerized device further comprises an illumination module configured to illuminate in response to an indication generated by the control system. In some cases, the computerized device further comprises a vibration system utilized to indicate on alerts to said person.

In some cases, the device is incorporated in a wearable device. In some cases, the device is water resistance. In some cases, the device further comprises a camera. In some cases, the camera is utilized to capture digital images on specific parts of the patient body. In some cases, the camera is utilized to capture digital images of the patient's physical position.

In some cases, the control system further comprises a security module to protect said person identity.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a plurality of sensors and other system components which can be integrated with a personal monitoring device (PMD), according to exemplary embodiment of the present invention.

FIG. 2 shows PMD components assembled on a pliable band, according to exemplary embodiment of the present invention.

FIG. 3 discloses a block diagram of the PMD, the analysis system and additional components which can be utilized in the measuring processes, according to exemplary embodiments of the present invention.

FIG. 4 shows PMD components incorporated into a pliable bracelet, according to exemplary embodiment of the present invention.

FIG. 5 shows PMD components incorporated into a footwear item, according to exemplary embodiment of the present invention.

FIG. 6 shows PMD components incorporated into a sandal, according to exemplary embodiment of the present invention.

FIG. 7 shows PMD components incorporated into an underwear, according to exemplary embodiment of the present invention.

FIG. 8 shows PMD components incorporated in a pliable band which can be worn by a person, according to exemplary embodiment of the present invention.

FIG. 9, discloses a method of receiving indicator, analyze them and identify the blood pressure patterns and respond in case an irregular pattern was found, according to exemplary embodiment of the present invention.

### DETAILED DESCRIPTION OF THE INVENTION

The present invention discloses method for utilizing a personal long-term pattern blood pressure monitoring device (PMD), designed to detect physiological indicators and conclude trends in the continuous blood pressure of a person. The PMD can be configured to detect a wide range of physiological indicators in order to achieve a prognosis of the blood pressure pattern in a more accurate manner. Thus, the PMD measures blood pressure of a specific person over time and sends the collected measurements to an analysis system that detects an abnormal trend in the collected information over time. The analysis system may reside in the PMD or in a remote device.

The PMD may be designed to integrate a plurality of sensor types which can be utilized to measure diverse physiological indicators in the human body, accumulate the physiological indicators and thereby conclude the current blood pressure status and the blood pressure trends of a living subject such as a human body. Such physiological sensors utilized by the PMD can be: Blood pressure sensor, heart-rate sensor, blood oxygen sensor, glucose sensor, body heat sensor, skin color and/or skin condition, lung capacity sensor, blood-flow rate sensor, voice detection and the like. In some cases, the PMD may also be designed to integrate additional sensors such as vibration sensor, skin electricity sensor, tilt and/or movement sensors, and the like. In some embodiments of the present invention, the PMD may be configured to operate with an analysis system designed to receive the physiological indicators measured by the sensors and perform diverse processes to conclude the current blood pressure status and the blood pressure trends of a living subject such as a human body. In some cases, such analysis system may be a computer program or a batch of computer programs which can operate a plurality of analysis processes designed to facilitate the analysis operation. The analysis processes may also be configured to monitor changes in the user's blood pressure and compare the physiological indicators with the corresponding indicators gathered from general population. In some cases, the analysis system can be configured to communicate with external systems in order to receive and utilize statistical data gathered from multiple people to determine the initial baseline for a given person which uses the PMD. Such external systems may be a database comprising information on people, a third party server or a service which provides such information, and the like. In some cases, some data needed for analyzing the data may be inserted manually by a person. Such data may be statistic information, common trends in the general population, and the like.

In some cases, the analysis processes may also comprise processes such as comparing newly collected data with historical data, execute business intelligent processes to identify trends, comparing newly collected data with known patterns in order to evaluate the medical condition, the physiological conditions, the blood pressure trends, and the like. Such data may be blood pressure information, information collected from the physiological sensors, information collected from the environmental sensors and the like. The present invention also discloses a method of correlating information collected from the physiological sensors and from the environmental sensors associated with a specific person with prior blood pressure measurements of the person to determine or update the person's blood pressure trends.

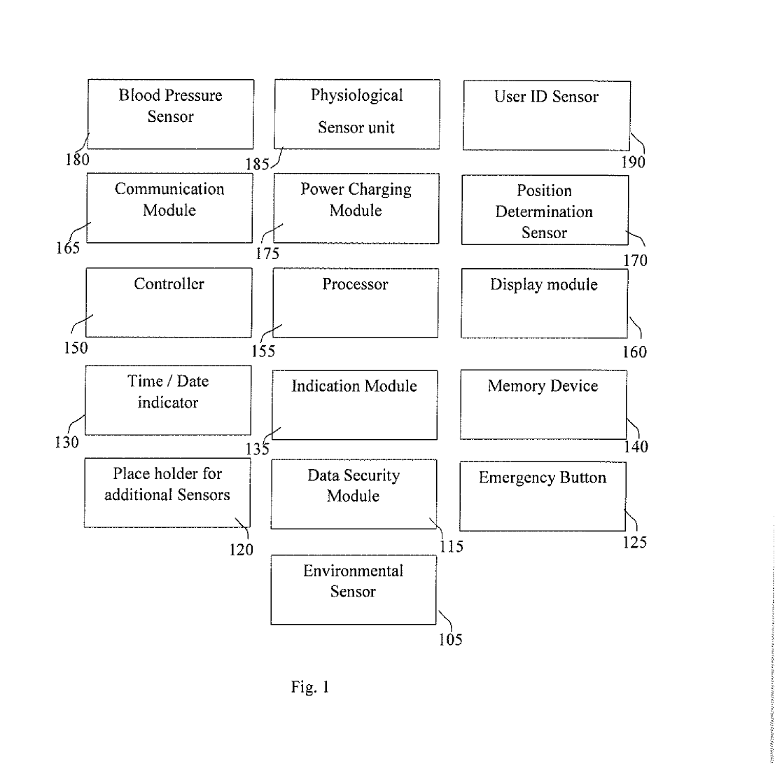

FIG. 1 shows a plurality of sensors and other system components which can be integrated with the PMD, according to exemplary embodiment of the present invention. The PMD may comprise a Blood Pressure Sensor 180 designed to measure the blood pressure in the user's body. In some cases, the blood pressure sensor 180 may be a non-invasive sensor. The PMD may also comprise a physiological sensor unit 185 which may comprise at least one sensor such as, hart-rate sensor, blood oxygen sensor, glucose sensor, body heat sensor, vibration sensor, skin electricity sensor, skin color and/or skin condition (such as, but not limited to, wet, dry, etc. . . . ) sensors, a lung capacity sensor, voice detection and/or identification sensor, and the like. The PMD may also comprise a User ID sensor 190 designed to possess the identity credentials of the person. In some cases, the credential may be utilized to identify the person and associate the indicators and/or the data measured by the PMD sensors to a specific person. For example, in case a blood pressure is measured in a person's body, the blood pressure measurements are transmitted to a remote computer via the internet. In such case, the remote computer may be configured to associate the transmitted data with the person's ID. The ID, or the identify credential, may be held by the User ID sensor 190. In some embodiments of the present invention, the User ID sensor 190 may be able to hold some biological information which may be utilized in order to identify the person. Such biological information may be a finger print digital sample, an iris digital, a digital sample for voice recognition process, and the like.

In some cases, the PMD may also comprise a communication module 165 designed to communicate with other components. Such components may be said analysis system, a database located in a communications network or on the internet, and the like. The communication module 165 may comprise a wireless module to communicate wirelessly with servers or databases located in the communications network. Such wireless module may be a module capable to communicate via Wi-Fi, telecommunication network, wireless devices such as Bluetooth, IR, and the like. In some cases, the communication module 165 may comprise a device capable to communicate via a wired network such as LAN, ADSL, MAN, WAN, and the like. The PMD may also comprise a power charging module 175 utilized to deliver the electrical power needed to operate the PMD. In some cases, the power charging module 175 may comprise chargeable batteries and a socket to plug in the power charger cable. The power charging module 175 may also comprise diverse charger types such as a charger using the person's movement to charge the power source, a solar charger, or a wireless charger. In some cases, the power charging module 175 may be able to comprise non-rechargeable batteries. In some other cases, the power charging module 175 may be designed to utilize a power cable only without batteries.

The PMD may also comprise a position determination sensor 170 designed to host position sensors and to transmit the captured information regarding the person's position to the analysis system. The position determination sensor 170 may host additional sensors such as a tilt sensor, sensors which detect the person's movement, a device that measures acceleration, a gyroscope, and the like. The PMD may also comprise a controller 150 configured to interface with the PMD components and control the data flow among the PMD components of the device. For example, in case the blood pressure sensor 180 is configured to transmit data to the analysis system or to a server via communication module 165, the controller 150 may detect it and allow the communication between the blood pressure sensor 180 and the communication module 165. In such cases, the processor 155 may be involved to process the data, prepare it and locate it in the memory device 140 in order to facilitate the communication via the communication module 165. Then, the communication module 165 may transmit the data from the memory device 140 to a server or to the analysis system as elaborated above. In some cases, the PMD may also comprise a display 160 which allows displaying information to the PMD user. Such information may be indication or alert on a blood pressure status or trend, visual representation of the indicators collected by the sensors, system information data such configuration status of the PMD components, and the like. For example, in case the blood pressure sensor 180 is configured to transmit data to a server or to the analysis system via communication module 165, the PMD user may be able to use the display 160 to see information such as the time the blood pressure sensor 180 has started to collect the data, the amount of collected data, the address of the server to which the data is transmitted to and the like. In some cases, the display 160 may be able to communicate via communication module 165 to the server or to the analysis system and display the analyzed data from the server. Such analyzed data may be the person's blood pressure trends, the blood pressure current status, additional information collected by other sensors such as sensors integrated to the physiological sensor 185, and the like.

The PMD may also comprise an indication module 135 designed to integrate indication devices to notify or alert the PMD user or other electronic devices of persons defined as receiving notifications concerning the person's blood pressure status and/or trends, such as the person's medical doctor or physicians. The indication module 135 may also comprise a vibration device which can vibrate in case the blood pressure trend reaches a certain threshold, an additional visual device, a sound device which creates alerts in case a specific threshold is reached, and the like. The PMD may also comprise a data security module 115 which manages activities in order to enhance and maintain the security level of the PMD. Such security activities may be encrypting the collected data, encrypting the communication between the PMD and the analysis server, and the like. The PMD may also comprise and emergency button 125 utilized in case an emergency act is required. The emergency acts may be call the insurance company, call the regular family physician, call a family member, and the like.

The PMD may also comprise a time/date indicator 130 configured to communicate with the blood pressure sensor 180 and with the physiological sensor 185 and provide the current time associated with a sensor's measurement. In some cases, the time/date indicator 130 may be used to present the current time on the display 160. The time/date indicator 130 may also have the option to track the time and date to other components of the PMD. For example, the position deamination 170 may receive the information about the current time and date and the information to the data regards the person position. The data may be transmitted to a server or to the analysis system and used to analysis the position changing in relation to the time in the day. The PMD may also comprise an environmental sensor 105 designed to collect data regards the environmental conditions at the vicinity of the PMD user. Such environmental sensor may be a barometer, temperature sensor, humidity sensor, air pollution sensor, and the like. The data on the environmental conditions may be transmitted to the analysis system in order to be included in the analysis processes conducted by the analysis system. In some cases, the PMD may include a place holder 120 which physically enables hosting or physical connection to additional sensors. The additional sensors may be an additional environmental sensor, an additional physiological sensor, an additional blood pressure sensor, and the like. In some embodiments of the present invention, the PMD may be a water resistance device, and in some cases, integrated with a camera, or a microphone.

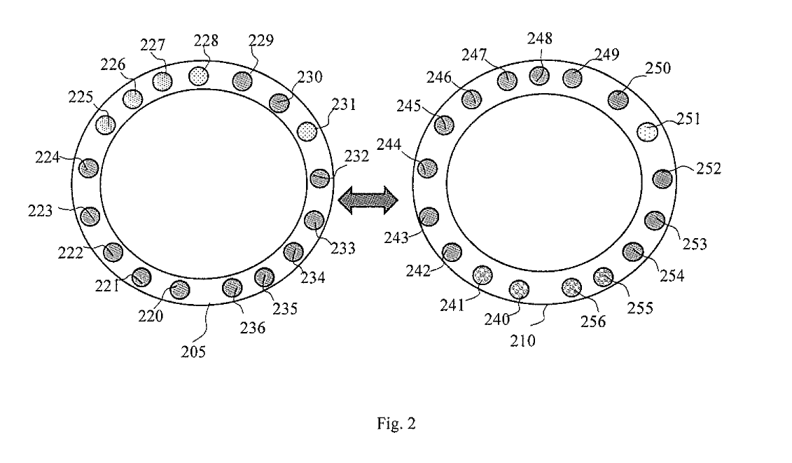

FIG. 2 shows PMD components assembled on a pliable band, according to exemplary embodiment of the present invention. PMD band 205 comprises a plurality of sensors and PMD components 220, 221, 222, 223, 224, 225, 226, 227, 228, 229, 230, 231, 232, 233, 234, 235, and 236. Such a plurality of sensors and PMD components may include a blood pressure sensor, physiological sensors, environmental sensors, and additional sensors as described above. In some cases, the sensors and PMD components integrated with the PMD band 205 may be designed to change colors in case detecting predefined thresholds. The predefined thresholds may be configured in or identified by the analysis system. The predefined thresholds may refer to blood pressure status or trends. For example, in case a sensor 235 which can be any blood pressure sensor or a physiological sensor, as described above, detects a measurement that is higher or lower than a predefined threshold, the sensor 235 may change its or a color of an indication module in order to indicate and alert the PMD user. In some cases, the sensors and PMD components may be configured to vibrate emit a sound in case the predefined threshold is reached.

FIG. 2 also shows a PMD band 210 that comprises a plurality of sensors and PMD components 240, 241, 242, 243, 244, 245, 246, 247, 248, 249, 250, 251, 252, 253, 254, 255, and 256. PMD band 210 shows different status of the sensors and PMD components which presented in different colors. For example, sensor 228 integrated in PMD band 205 is presented in a white dotted color. Sensor 228 may be a blood pressure sensor as elaborated above. Sensor 248 integrated into the PMD band 210, which may be the same type as sensor 228 but presented in a gray dotted color. The change in colors and presentation type may be result from a different level of blood pressures measured in each of the sensors. Such presentation may be dynamic and change in response to the measured values. In some cases, a vibration or a sound may be generated or emitted by the indication module 135, according to a predefined set of rules.

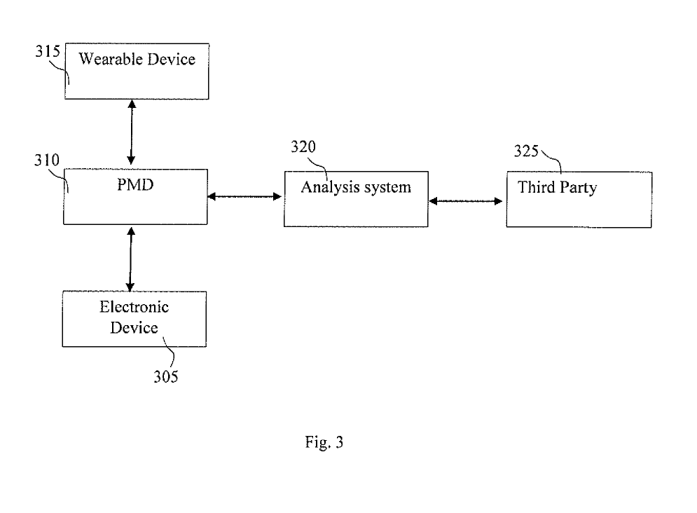

FIG. 3 discloses a block diagram of the PMD, the analysis system and additional components that can be utilized in the measuring processes, according to exemplary embodiments of the present invention. PMD 310 is designed to collect blood pressure measurements over time, for example a week to 15 years. The collected blood pressure measurements are later used to detect personal long-term pattern in the blood pressure of a living subject such as a human body, according to the method disclosed in the present invention. The PMD 310 may be attached to a user so the sensors may be in a physical contact to the monitored person's body. In some cases, the sensors may be fastened to the person's body using a band, a bracelet, a rubber, a flexible garment item, and the like. The PMD 310 may detect indicators associated with the measured blood pressure, the physiological conditions, and in some cases, the environmental conditions, and transmit the data to the analysis system 320. The analysis system may be operated on a computerized device designed to carry out computerized processes. Such a computerized device can be a computerized server, a virtual server, a personal computer, a tablet personal computer, a cluster of servers, and the like. In some cases, the analysis system 320 may comprise a database configured to collect the indicators transferred from the PMD 310 and store it. The analysis system 320 may analyze the collected blood pressure measurements according to a predefined rule, or algorithms. Such rules may be “if the collected blood pressure is higher than last month measurement by 10 percent, notify the person's doctor”.

In some cases, the analysis system 320 may also comprise algorithms, computerized processes, business intelligence procedures, and the like, in order to analyze the collected measurements and data, and detect trends and patterns in the continuous blood pressure of the user who carries the PMD 310. For example, a transmitted of the PMD transmits data regarding the blood pressure to the analysis system 320. The analysis system 320 captures the data, compares it to patterns identified in the general population and thus identify pattern in the blood pressure measurements over time. The analysis system 320 can be configured to send alerts or/and notifications in case the identified patterns of the user meet any predefined thresholds. The analysis system 320 may also store the pre-identified patterns of a plurality of persons using the analysis system 320 and utilize them as a baseline patterns for identifying a blood pressure pattern of the PMD user. The analysis system 320 may also be able to associate data captured by the PMD with users' identity. In some cases, the PMD may also have an identity stored in the analysis system 320.

The analysis system 320 may be able to collect the data transferred from other sensors such as the physiological sensors or/and the environmental sensors, add it to the analysis processes and change the measuring settings if needed. For example, the analysis system 320 may utilize the movement sensors and the heartbeat sensor to identify that the user carries the PMD may be running, or walking fast, and identify whether the user stands, sits or lies on a bed. In such cases, the analysis system 320 may change the measuring frequency and run the analysis processes in different timing. For example, in case the analysis system 320 detects that the person carries the PMD is running, the blood pressure may be analyzed in a higher frequency than in cases the person carries the PMD is detected as resting. In some cases, the analysis system 320 may be configured to associate more than one baseline pattern with a person. For example, the analysis system may associate a blood pressure baseline pattern with a person in case the person is running, another blood pressure baseline pattern may be associated with the person in case the person is walking, and another baseline pattern may be associated with the same person in case the person is resting.

The analysis system 320 may be able to receive the data, analyze the user's movement and utilize the corresponded baseline pattern for the user status. In some cases, the analysis process performed by the analysis system 320 may utilize additional data captured by the PMD. Such additional data may be the time in the day, the user's gender, the level of specific hormones in the user's body, the user's level of sweat and the like. The analysis system 320 may also add external information to the analysis process such as medications taken on a regular basis, and/or the bodily part in which the blood pressure measurement was performed.

In some embodiments of the present invention, the analysis system 320 may be designed to identify patterns in the blood pressure and then determine if the identified patterns can be defined as regular patterns or irregular patterns. The pattern determination may be performed by comparing the identified patterns with predefined thresholds. In case the analysis system 320 identified patterns as irregular patterns, the analysis system 320 may provide alerts or notifications regarding the defined findings of the irregular patterns. Such alerts and/or the notifications may be addressed to the health maintenance organization, to the insurance company, to the user, to the primary care physician, or to other persons or organizations as defined in the analysis system 320.

In some exemplary embodiments of the present invention, the PMD 310 may communicate with another wearable device 315. Such wearable device 315 may be another PMD, an external physiological sensor, environmental sensor integrated in the garments of the person and the like. The PMD 310 may also be able to connect with electronic device 305 associated with the PMD user. The electronic device 305 may be an electronic watch, a computerized mobile device, such as a mobile telephone or a tablet personal computer, and the like. In some cases, the PMD 310 may be configured to send the collected data to the electronic device 305 in order to present it to the user. Thus, the electronic device 305 may receive the indicators and present them at the local display of the electronic device 305. In some cases, the analysis system 320 may also be configured to transmit data captured by PMDs such as PMD 310 to a third party server. In some cases, the analysis system 320 may also be configured to carry out administrative work such as define new users, configure the addressees of the entity which receive the information, and the like.

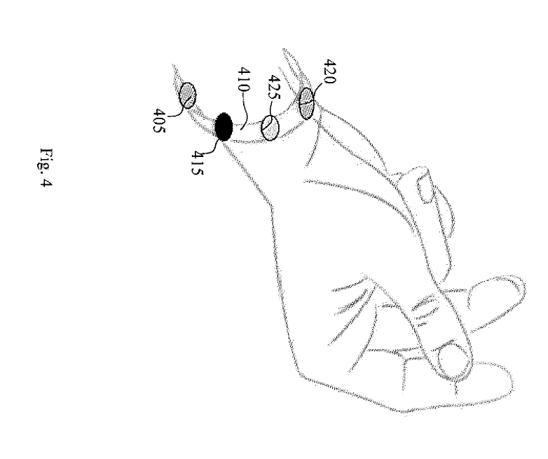

FIG. 4 shows PMD components incorporated into a pliable bracelet, according to exemplary embodiments of the present invention. FIG. 4 shows a possible embodiment of the PMD embedded in a bracelet 410, which can be worn around the wrist or the ankle, and comprising a plurality of sensors and PMD components. Bracelet 410 comprises sensors 420, 425, 415, and 405 that can be blood pressure sensors, physiological sensors, environmental sensors, and the like, as described above. In some cases, the bracelet 410 can be designed in a form of a finger ring device worn on the user's finger. The bracelet 410 may be designed to change colors in case meeting specific thresholds configured in the analysis system. In some embodiments of the present invention, the PMD can be incorporated into a watch strap in a similar fashion as incorporated into bracelet 410. In such possible embodiments, the watch can be a computerized also known as a smartwatch and the PMD may be configured to transmit the indicators captured by the sensors to the smartwatch. In some cases, the smartwatch may be configured to operate the analysis system.

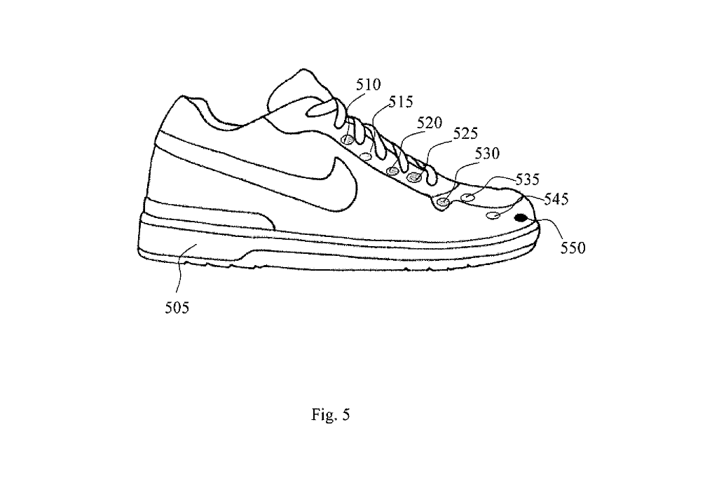

FIG. 5 shows PMD components incorporated into a footwear item, according to exemplary embodiment of the present invention. Shoe 505 is integrated with a plurality of sensors and PMD components. Shoe 505 comprises sensors 510, 515, 520, 525, 520, 535, 545, and 550 which can be blood pressure sensors, physiological sensors, environmental sensors, and the like, as described above. In some cases, the sensors and PMD components integrated with the shoe 505 may be designed to change colors in case the analysis system detects an irregular blood pressure pattern or in case the measured blood pressure reaches predefined thresholds.

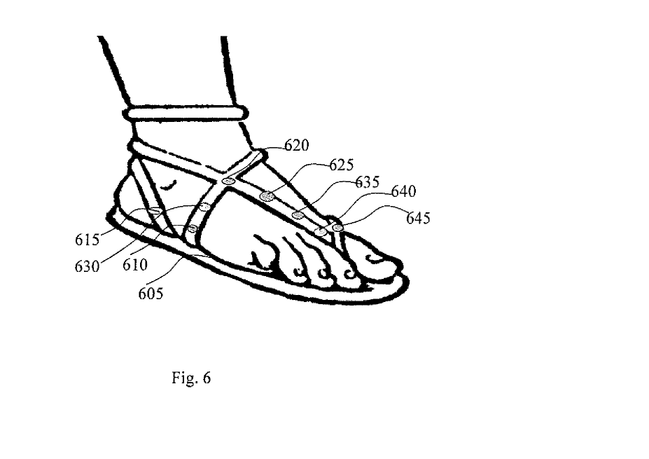

FIG. 6 shows PMD components incorporated into a sandal, according to exemplary embodiment of the present invention. Sandal 605 is integrated with a plurality of sensors and PMD components. Sandal 605 comprises sensors 610, 615, 620, 625, 620, 635, 640, and 645 which can be a blood pressure sensor, physiological sensors, environmental sensors, and the like, as described above. In some cases, the PMD components may be incorporated into sandals, socks, sneakers, or other footwear items.

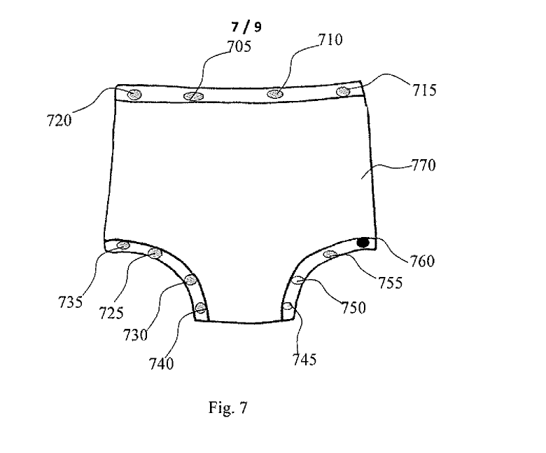

FIG. 7 shows PMD components incorporated into an underwear, according to exemplary embodiment of the present invention. FIG. 7 shows an underwear 770 integrated with a plurality of sensors and PMD components. Underwear 770 comprises sensors 705, 710, 715, 720, 725, 730 735, 740, 745, 750, 755 and 760 which can be blood pressure sensors, physiological sensors, environmental sensors, and the like, as described above. In some cases, the PMD components can be incorporated into a dedicated garment item, such as a shirt/pants sleeve, and the like. In some embodiments of the present invention the PMD, may be incorporated into a textile material which can be worn by a person.

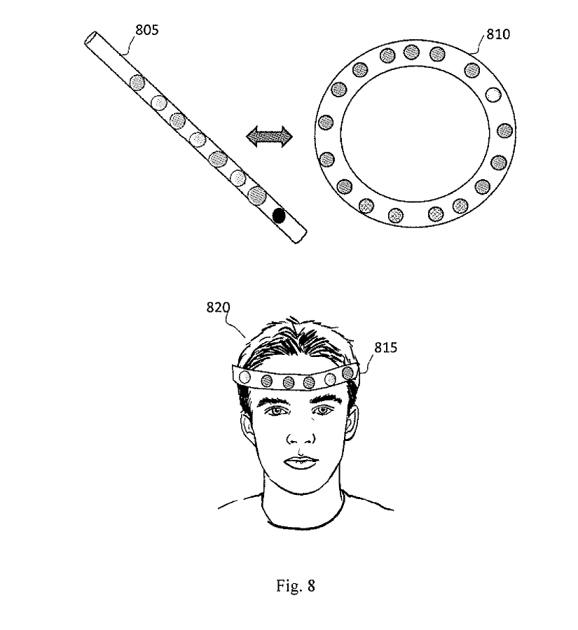

FIG. 8 shows PMD components incorporated in a pliable band which can be worn by a person, according to exemplary embodiment of the present invention. FIG. 8 shows a possible embodiment of the PMD embedded in a head band 805 and comprising a plurality of sensors and PMD components. Band 805 comprises sensors which can be a blood pressure sensor, physiological sensors, environmental sensors, and the like, as described above. Band 805 can be designed in a form of a circular band 810 configured to surround the user's head 820. The circular band 815 may be designed to change colors in case meeting specific thresholds configured in the analysis system. In some embodiments, the PMD implemented in circular band 815 may be configured to transmit the data captured by the sensor to the smartwatch, or to a mobile telephone device.

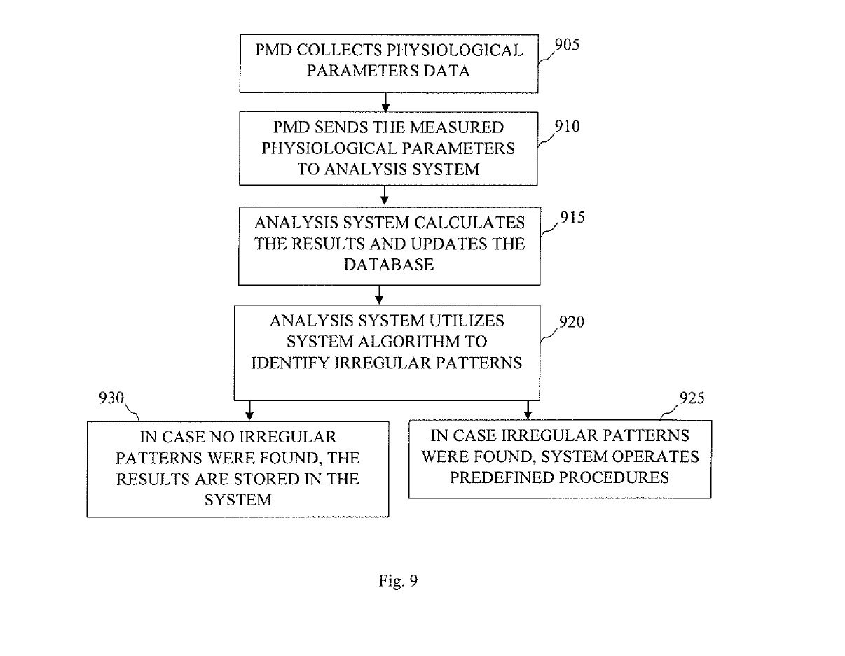

FIG. 9, discloses a method of receiving indicators, analyze them and identify the blood pressure patterns and respond in case an irregular pattern was found, according to exemplary embodiment of the present invention. In step 905, a PMD collects the blood pressure rate and some physiological parameters. In step 910, the PMD sends the measured physiological parameters and the blood pressure rate to the analysis system. In some cases, the PMD may send only the blood pressure rate, or send the physiological parameters without the blood pressure rate, for example according to predefined procedures controlled by the analysis system. The PMD may also send indicators provided from environmental sensors and data regarding the user's position and/or location, the user's movement and the like. In step 915, the analysis system receives the data, calculates the results and updates the database. In step 920, the analysis system utilizes system algorithm to identify irregular patterns. Such irregular patterns may be identified by comparing the calculated results with the user's history, comparing the calculated results with data taken from the general public, or any other thresholds defined in the analysis system.

In step 925, the analysis system may identify irregular patterns and operate predefined procedures. The predefined procedures may include sending alerts and/or the notifications to the person who carries the PMD to the health maintenance organization, to the insurance company, to the primary care physician, or to other persons or organizations as defined in the analysis system. In some cases, the analysis system may operate the predefined procedures repeatedly, a plurality of times. The analysis system may also store the data associated with the irregular patterns in a database and associate them with the person who carries the PMD. Step 930 shows a case where no irregular patterns were found. In such case, the analysis system may store the data that regards the regular pattern in a database and associate it with the person who carries the PMD.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A method, comprising:
collecting information from at least one blood pressure sensor attached to a person, wherein said collected information is related to the blood pressure conditions of the person;
transmitting said collected information to an analysis system, said analysis system has access to prior blood pressure measurements of the person;
transmitting the person's identity to said analysis system, the person is associated with said transmitted collected information;
comparing said collected information with historical blood pressure information of said person;
detecting an abnormal trend according to said comparison of the collected information with the historical information related to the blood pressure conditions of said person;
generating an indication according to predefined procedures in case the abnormal trend is detected, and;
storing said newly collected information in the analysis system.

2. The method of claim 1, further comprises transmitting a date and a time of said information collection to the analysis system.

3. The method of claim 1, further comprises transmitting a physical position of said person during collection of the information to the analysis system.

4. The method of claim 1, further comprises collecting additional physiological data of the person.

5. The method of claim 1, further comprises transmitting information about the person's movements during collection of the information to the analysis system.

6. The method of claim 1, further comprises securing the identity of the person associated with the collected blood pressure information.

7. A computerized device integrated with at least one blood pressure sensor, comprising:
an independent power source;
a processor to collect blood pressure information from the at least one blood pressure sensor;
a memory unit to store the information collected by the processor;
a communication device, and;
a control system designed to associate the collected information with the person identity, transmit the collected information to a remote analysis system, receive feedback from said remote analysis system, and provide an indication to said person according to said received feedback.

8. The computerized device of claim 7, further comprises sensors for sensing the physical position of said person while collecting the blood pressure information.

9. The computerized device of claim 7, further comprises a display unit.

10. The computerized device of claim 9, wherein the display unit is used by said control system to display alerts to said person.

11. The computerized device of claim 7, wherein the communication device is a wireless communication device.
The computerized device of claim 7, wherein the communication device is a wired communication device.

12. The computerized device of claim 7, further comprises an illumination module configured to illuminate in response to an indication generated by the control system.

13. The computerized device of claim 7, further comprises a vibration system utilized to indicate on alerts to said person.

14. The computerized device of claim 9, wherein the device is incorporated in a wearable device.

15. The computerized device of claim 9, wherein the device is water resistance.

16. The computerized device of claim 9, further comprising a camera.

17. The computerized device of claim 16, wherein the camera is utilized to capture digital images on specific parts of the patient body.

18. The computerized device of claim 16, wherein the camera is utilized to capture digital images of the patient's physical position.

19. The computerized device of claim 7, wherein the control system further comprises a security module to protect said person identity.
